(19) United States
(12) Patent Application Publication
(10) Pub. No.: US 2024/0081666 A1
(43) Pub. Date: Mar. 14, 2024

(54) TREND LINES FOR SEQUENTIAL PHYSIOLOGICAL MEASUREMENTS OF VESSELS

(71) Applicant: BOSTON SCIENTIFIC SCIMED, INC., Maple Grove, MN (US)

(72) Inventors: Wenguang Li, Los Gatos, CA (US); Marc Stepkowski, Livermore, CA (US)

(73) Assignee: BOSTON SCIENTIFIC SCIMED, INC., Maple Grove, MN (US)

(21) Appl. No.: 18/466,640

(22) Filed: Sep. 13, 2023

Related U.S. Application Data
(60) Provisional application No. 63/375,571, filed on Sep. 14, 2022.

(51) Int. Cl. A61B 5/0215; A61B 5/00; A61B 5/02; A61B 5/026

(52) U.S. Cl. CPC A61B 5/02154; A61B 5/0004; A61B 5/02007; A61B 5/026; A61B 5/6851; A61B 5/7246; A61B 5/7275; A61B 5/7278; A61B 5/742

(57) ABSTRACT

The present disclosure provides to identify regions of a cardiac cycle based on pressure measured intravenously during a procedure and to derive a fractional flow reserve from the measured pressure during the identified regions. Further the disclosure provides to derive a trend line of the derived fractional flow reserve while the patient in undergoing a procedure, such as a pre-percutaneous coronary intervention.

### CROSS-REFERENCE TO RELATED APPLICATIONS

This application claims the benefit of U.S. Provisional Patent Application Ser. No. 63/375,571 filed on Sep. 14, 2022, the disclosure of which is incorporated herein by reference.

### TECHNICAL FIELD

The present disclosure generally relates to hemodynamic systems. Particularly, but not exclusively, the present disclosure relates to assessing fractional flow reserves.

### BACKGROUND

Numerous tools are on the market to treat vascular lesions including plain old balloon angioplasty (POBA), atherectomy, cutting or scoring balloons, high pressure balloons, and percutaneous coronary intervention (PCI). PCI is a non-surgical procedure where a stent is placed inside a vessel at a location of a stricture using a catheter to increase the size of the lumen in the area of the structure. Prior to placing the stent, various physiological assessments of the vessel are made. For example, a pressure guide wire can be inserted into the vessel and pulled back (e.g., through the region of interest) to a guide catheter while continuously measuring pressure. From this measured pressure, several pressure rations can be derived.

However, interpretation of the pressure ratios, even when the ratios are plotted is challenging. Thus, there is a need for an improved method to derive, plot, and present pressure rations for interpretation by a physician prior to placing a stent as part of a PCI.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides trend lines to assess fractional flow reserves from distal and aortic pressures measured during an intravenous pullback operation.

In some implementations, the present disclosure be embodied as a system, for example, a system for determining fractional flow reserve (FFR) in the absence of administering a hyperemic agent, the system comprising a memory device comprising instructions; and a processor coupled to the memory device, the processor configured to execute the instructions, which when executed cause the system to: receive an indication of a first pressure from a pressure sensing guidewire coupled to the system for a first time period, wherein the first time period is during a medical intervention and wherein the first pressure is measured within a vessel of a patient; receive an indication of a second pressure from a pressure sensing medical device different from the pressure sensing guidewire for the first time period, wherein the second pressure is measured within the vessel of the patient; calculate a slope of a plot of the second pressure over the first time period; calculate a mean of the second pressure over the first time period; identify one or more regions within the first time period where the second pressure is less than or equal to the mean of the second pressure and where the slope of the plot of the second pressure is less than or equal to zero; calculate a plurality of FFR values as the ratio of the first pressure over the second pressure during the one or more regions; identify for each of the plurality of FFR values, a maximum of the FFR value and n prior FFR values where n is greater than one; and generate a trend line comprising the identified maximum FFR values.

Alternatively, or additionally in any of the embodiments of a system above, n is greater than or equal to 3.

Alternatively, or additionally in any of the embodiments of a system above, n is greater than or equal to 3 and less than or equal to 5.

Alternatively, or additionally in any of the embodiments of an apparatus above, the memory device can further comprise instructions that when executed by the processor cause the system to identify the one or more regions within the first period where the second pressure is less than or equal to a factor of the mean.

Alternatively, or additionally in any of the embodiments of a system above, the factor is between 0.75 and 0.85.

Alternatively, or additionally in any of the embodiments of a system above, the factor is 0.8.

Alternatively, or additionally in any of the embodiments of a system above, the pressure sensing guidewire comprises an optical pressure sensor.

Alternatively, or additionally in any of the embodiments of a system above, the pressure sensing medical device comprises a catheter with a pressure sensor.

Alternatively, or additionally in any of the embodiments of a system above, the pressure sending guidewire and/or the pressure sensing medical device are wirelessly coupled to the system.

Alternatively, or additionally in any of the embodiments of a system above, the system comprises the pressure sending medical device and the pressure sensing guidewire.

Alternatively, or additionally in any of the embodiments of a system above, the pressure sensing guidewire is configured to be disposed in the vessel distal of an intravascular lesion.

Alternatively, or additionally in any of the embodiments of a system above, the pressure sensing medical device is configured to be disposed in the vessel proximal of the intravascular lesion.

Alternatively, or additionally in any of the embodiments of an apparatus above, the memory device can further comprise instructions that when executed by the processor cause the system to generate a graphical user interface comprising a graphical representation of the trend line.

Alternatively, or additionally in any of the embodiments of an apparatus above, the memory device can further comprise instructions that when executed by the processor cause the system to render the graphical user interface for display on a display.

Alternatively, or additionally in any of the embodiments of an apparatus above, the system comprising the display.

In some implementations, the present disclosure be embodied as a method, for example, a method for determining fractional flow reserve (FFR) in the absence of administering a hyperemic agent, the system comprising receiving an indication of a first pressure from a pressure sensing guidewire coupled to the system for a first time period, wherein the first time period is during a medical intervention and wherein the first pressure is measured within a vessel of a patient; receiving an indication of a second pressure from a pressure sensing medical device different from the pressure sensing guidewire for the first time period, wherein the second pressure is measured within the vessel of the patient; calculating a slope of a plot of the second pressure over the first time period; calculating a mean of the second pressure over the first time period; identifying one or more regions within the first time period where the second pressure is less than or equal to the mean of the second pressure and where the slope of the plot of the second pressure is less than or equal to zero; calculating a plurality of FFR values as the ratio of the first pressure over the second pressure during the one or more regions; identifying for each of the plurality of FFR values, a maximum of the FFR value and n prior FFR values where n is greater than one; and generating a trend line comprising the identified maximum FFR values.

Alternatively, or additionally in any of the embodiments of a method above, n is greater than or equal to 3.

Alternatively, or additionally in any of the embodiments of a method above, n is greater than or equal to 3 and less than or equal to 5.

Alternatively, or additionally in any of the embodiments of a method above, the method can comprise identifying the one or more regions within the first period where the second pressure is less than or equal to a factor of the mean.

Alternatively, or additionally in any of the embodiments of a method above, the factor is between 0.75 and 0.85.

Alternatively, or additionally in any of the embodiments of a method above, the factor is 0.8.

Alternatively, or additionally in any of the embodiments of a method above, the pressure sensing guidewire comprises an optical pressure sensor.

Alternatively, or additionally in any of the embodiments of a method above, the pressure sensing medical device comprises a catheter with a pressure sensor.

Alternatively, or additionally in any of the embodiments of a method above, the pressure sending guidewire and/or the pressure sensing medical device are wirelessly coupled to the system.

Alternatively, or additionally in any of the embodiments of a method above, the pressure sensing guidewire is configured to be disposed in the vessel distal of an intravascular lesion.

Alternatively, or additionally in any of the embodiments of a method above, the pressure sensing medical device is configured to be disposed in the vessel proximal of the intravascular lesion.

Alternatively, or additionally in any of the embodiments of a method above, the method can comprise generating a graphical user interface comprising a graphical representation of the trend line.

Alternatively, or additionally in any of the embodiments of a method above, the method can comprise rendering the graphical user interface for display on a display.

In some implementations, the present disclosure be embodied as an apparatus for determining fractional flow reserve (FFR) in the absence of administering a hyperemic agent, comprising a processor coupled to a memory, the memory comprising instructions executable by the processor, the processor configured to execute the instructions, which instructions when executed cause the processor to implement the method of any combination of the examples above.

In some implementations, the present disclosure be embodied as at least one machine readable storage device, comprising a plurality of instructions that in response to being executed by a processor of system for determining fractional flow reserve (FFR) in the absence of administering a hyperemic agent cause the processor to implement the method of any combination of the examples above.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates a pressure measurement system.

FIG. 2 illustrates a logic flow for a pressure measurement system.

FIG. 3 illustrates a plot of measured pressure.

FIG. 4A illustrates a plot of measured pressure over a period including flushing events.

FIG. 4B illustrates a plot of a trend line over the period of the plot in FIG. 4A.

FIG. 5 illustrates a plot of a trend line versus an FFR.

FIG. 6 illustrates a computer-readable storage medium.

FIG. 7 illustrates a diagrammatic representation of a machine.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

During some medical interventions (e.g., pre-percutaneous coronary intervention (pre-PCI), it may be desirable to measure and/or monitor the blood pressure within a blood vessel. For example, some medical devices may include pressure sensors that allow a clinician to monitor blood pressure. Such devices may be useful in determining fractional flow reserve (FFR), which may be understood as a ratio of the pressure after or distal of a stenosis, referred to as the distal pressure (P_{d}) relative to the pressure before the stenosis or the aortic pressure (Pₐ). In other words, FFR may be understood as P_{d}/Pₐ.

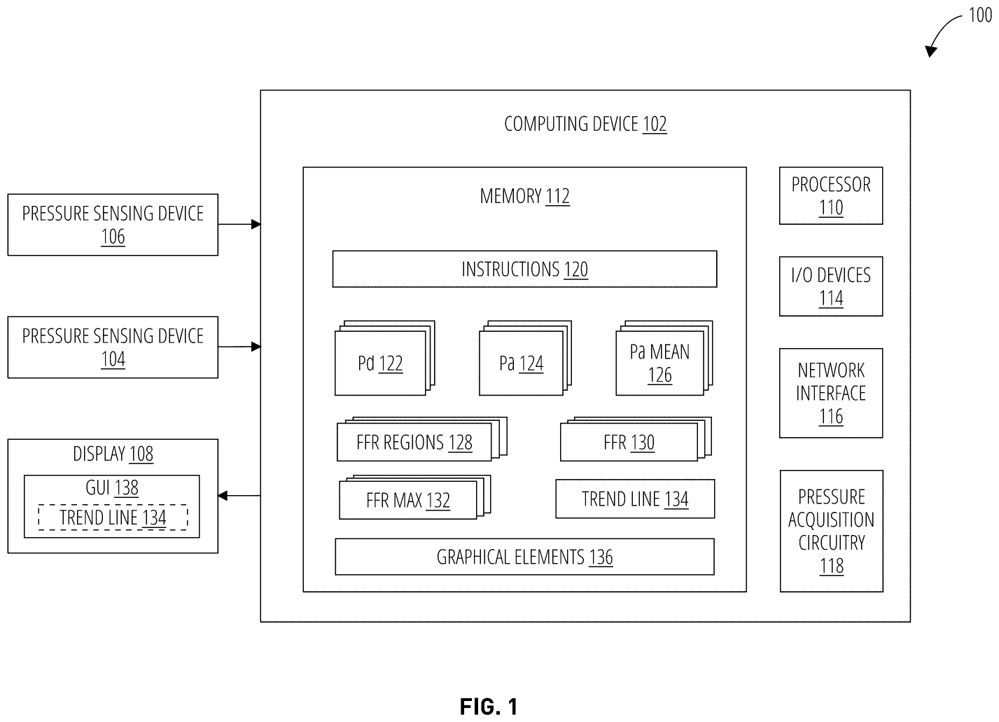

FIG. 1 illustrates a pressure measurement system 100, according to some embodiments of the present disclosure. In general, pressure measurement system 100 is a system for measuring P_{d} and Pₐ during a pre-PCI procedure, deriving a pressure ratio based on P_{d} and Pₐ and displaying a trend line of the pressure ratio during the pre-PCI procedure. Pressure measurement system 100 can be implemented in a commercial hemodynamic system, an intravascular guidance system, or intravascular ultrasound (IVUS) guidance, such as, for example, the AVVIGO® Guidance System available from Boston Scientific®.

The present disclosure provides advantages over prior or conventional pressure measurement systems in that the pressure ratio and trend lines are generated in real time and can be displayed during a pre-PCI procedure in a mechanism that is more easily digestible or interpretable than conventional pressure measurements. Further, often during a pre-PCI procedure, artifacts in the recording of the pressure can cause temporary fluctuations in the pressure readings. For example, flushing the catheter and/or vessel will cause artifacts in the pressure measurement. Further, pressure can be measured while the distal pressure measurement device is “pulled back” towards the proximal pressure measurement device. This leads to difficult to decipher pressure curves. The present disclosure provides a ratio and trend line that recovers from such artifacts and enables physicians to easily identify where pressure changes (e.g., due to a stenosis, lesion, or other defect in the vessel).

Pressure measurement system 100 includes a computing device 102, pressure sensing device 104, pressure sensing device 106, and display 108. In general, pressure sensing device 104 can be a pressure sensing guidewire. In other instances, the pressure sensing device 104 may be a catheter or other type of pressure sensing medical device. The pressure sensing device 104 may be configured to measure blood pressure distal of an intravascular stenosis, or said differently, to measure the distal pressure P_{d}. With some embodiments, pressure sensing device 106 can be a pressure sensing catheter. Whereas, in other embodiments, pressure sensing device 106 can be a pressure sensing guidewire, or other pressure sending device. The pressure sensing device 106 may be configured to measure blood pressure proximal of an intravascular stenosis, or said differently, to measure the aortic pressure Pₐ. The pressure sensing devices 104 and 106 can be coupled to computing device 102, for example, via a connection cable and input port (not shown).

Computing device 102 can be any of a variety of computing devices. In some embodiments, computing device 102 can be incorporated into and/or implemented by a console of display 108. With some embodiments, computing device 102 can be a workstation or server communicatively coupled to pressure sensing devices 104 and 106. With still other embodiments, computing device 102 can be provided by a cloud based computing device, such as, by a computing as a service system accessibly over a network (e.g., the Internet, an intranet, a wide area network, or the like). Computing device 102 can include processor 110, memory 112, input and/or output (I/O) devices 114, network interface 116, and pressure acquisition circuitry 118.

The processor 110 may include circuitry or processor logic, such as, for example, any of a variety of commercial processors. In some examples, processor 110 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 110 may include graphics processing portions and may include dedicated memory, multiple-threaded processing and/or some other parallel processing capability. In some examples, the processor 110 may be an application specific integrated circuit (ASIC) or a field programmable integrated circuit (FPGA).

The memory 112 may include logic, a portion of which includes arrays of integrated circuits, forming non-volatile memory to persistently store data or a combination of non-volatile memory and volatile memory. It is to be appreciated, that the memory 112 may be based on any of a variety of technologies. In particular, the arrays of integrated circuits included in memory 120 may be arranged to form one or more types of memory, such as, for example, dynamic random access memory (DRAM), NAND memory, NOR memory, or the like.

I/O devices 114 can be any of a variety of devices to receive input and/or provide output. For example, I/O devices 114 can include, a keyboard, a mouse, a joystick, a foot pedal, a display, a touch enabled display, a haptic feedback device, an LED, or the like.

Network interface 116 can include logic and/or features to support a communication interface. For example, network interface 116 may include one or more interfaces that operate according to various communication protocols or standards to communicate over direct or network communication links. Direct communications may occur via use of communication protocols or standards described in one or more industry standards (including progenies and variants). For example, network interface 116 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 116 can include logic and/or features to enable communication over a variety of wired or wireless network standards (e.g., 202.11 communication standards). For example, network interface 116 may be arranged to support wired communication protocols or standards, such as, Ethernet, or the like. As another example, network interface 116 may be arranged to support wireless communication protocols or standards, such as, for example, Wi-Fi, Bluetooth, ZigBee, LTE, 5G, or the like.

The pressure acquisition circuitry 118 may include circuitry including custom manufactured or specially programmed circuitry configured to receive or receive and send signals between pressure sensing devices 104 and 106 including indications of P_{d} and Pₐ.

Memory 112 can include instructions 120. During operation processor 110 can execute instructions 120 to cause computing device 102 to receive (e.g., from pressure sensing device 104, or the like) measurements of P_{d} and store the measured pressures as distal pressure 122 in memory 112. For example, processor 110 can execute instructions 120 to receive signals from pressure sensing device 104 comprising indications of pressure while pressure sensing device 104 is inserted into a vessel of a patient and disposed distal to a stenosis. Further, processor 110 can execute instructions 120 to cause computing device 102 to receive (e.g., from pressure sensing device 106, or the like) measurements of Pₐ and store the measured pressures as aortic pressure 124 in memory 112. For example, processor 110 can execute instructions 120 to receive signals from pressure sensing device 106 comprising indications of pressure while pressure sensing device 106 is inserted into a vessel of a patient and disposed proximal to a stenosis. In some embodiments, processor 110 can execute instructions 120 to receive signals from pressure sensing device 104 while pressure sensing device 104 is “pulled back” or retracted proximally towards or until pressure sensing device 106.

Often, when looking at pressure (e.g., P_{d} and Pₐ, or the like) during a pre-PCI procedure, it may be desirable to measure a change or drop in pressure across a stenosis while under a maximum flow condition (e.g., hyperemia). Thus, several interventions that are performed to assess FFR include the administration of hyperemic agents such as adenosine to cause maximum flow conditions. For several reasons (e.g., patient comfort, extended procedure time, technical challenges associated with mixing adenosine for intravascular administration, cost, etc.), it may be desirable to reduce the use of hyperemic agents. Pressure measurements performed under a resting condition conduction are typically referred as resting indices. An example of such a measurement is resting P_{d}/Pₐ in which the ratio is computed with data from the whole cardiac cycle. Disclosed herein are methods for assessing/determining FFR that can be performed in the absence of hyperemic agents including adenosine.

The present disclosure provides for assessing/determining FFR in a timely manner that enhances the comfort for the patient and that does not require unnecessary additional processes and/or synchronization, which as noted above in necessary for the specific practical application with which this disclosure is directed, namely measuring FFR during a pre-PCI procedure. It is to be appreciated that the maximum coronary flow occurs during the diastolic period of the cardiac cycle. Therefore, measurements of P_{d} and Pₐ during a diastolic period may provide a ratio closer to FFR (e.g., a better approximation of FFR) than that obtained from the whole cardiac cycle. In addition to resting P_{d}/Pₐ, some methods for assessing FFR may include computing P_{d} and Pₐ during time windows from the diastolic period. For example, some interventions such as instantaneous wave-free ratio and/or iFR™ may attempt to measure FFR during diastole. Such methods may require accurate measurement of waveform timing and/or synchronization with an ECG, which may complicate the process for assessing/determining FFR. Disclosed herein are methods for assessing/determining FFR by monitoring P_{d} and Pₐ during specific windows during the diastolic period of the cardiac cycle.

To that end, processor 110 can execute instructions 120 to derive trend line 134 from distal pressure 122 and aortic pressure 124, to generate graphical elements 136 from the trend line 134 comprising an indication of a trend line of trend line 134 and generate GUI 138 from graphical elements 136 and display GUI 138 on display 108. In general, processor 110 can execute instructions 120 to derive aortic pressure mean 126 from aortic pressure 124, identify FFR regions 128 from aortic pressure 124 and aortic pressure mean 126 and derive FFR values 130 from distal pressure 122 and aortic pressure 124 based on FFR regions 128. Further, processor 110 can execute instructions 120 to identify FFR maximum values 132 and generate trend line 134 from FFR maximum values 132. This is described in greater detail below. However, it is noted, as stated above, this process is performed in real-time, for example, during pre-PCI procedure. As such, computing device 102 is a specially programmed machine comprising circuitry arranged to couple to pressure sensing device 104 and pressure sensing device 106 and to generate trend line 134 while a patient is undergoing the procedure.

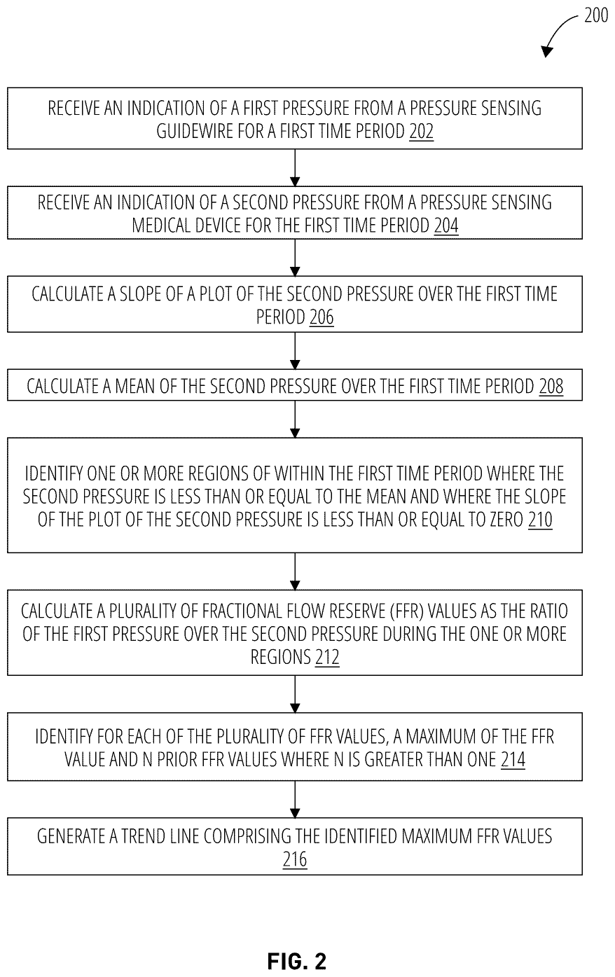

FIG. 2 illustrates a logic flow 200 to derive and plot a trend line of an FFR, according to some embodiments of the present disclosure. The logic flow 200 can be implemented by pressure measurement system 100 and will be described with reference to pressure measurement system 100 for clarity of presentation. However, it is noted that logic flow 200 could also be implemented by a hemodynamic system different than pressure measurement system 100.

Logic flow 200 can begin at block 202. At block 202 “receive an indication of a first pressure from a pressure sensing guidewire for a first time period” an indication of a first pressure for a first period is received from a pressure sensing guidewire. For example, a distal pressure can be received from a pressure sensing guidewire while the pressure sensing guidewire is disposed within a vessel of a patient and when the pressure sensing guidewire is retracted or pulled back towards a catheter. Processor 110 can execute instructions 120 to received distal pressure 122 from pressure sensing device 104 while pressure sensing device 104 is disposed in a vessel of a patient and pulled back towards pressure sensing device 106.

Continuing to block 204 “receive an indication of a second pressure from a pressure sensing medical device for the first time period” an indication of a second pressure for the first period is received from a pressure sensing medical device. For example, an aortic pressure can be received from a pressure sensing medical device while the pressure sensing medical device is disposed within the vessel of the patient and when a pressure sensing guidewire is retracted or pulled back towards the pressure sensing medical device. Processor 110 can execute instructions 120 to received aortic pressure 124 from pressure sensing device 106 while pressure sensing device 106 is disposed in a vessel of a patient and while pressure sensing device 104 is pulled back towards pressure sensing device 106.

In the example given above with respect to block 202 and block 204, pressure sensing device 104 can be a pressure sensing guidewire disposed in the vessel through pressure sensing device 106, which itself can be a pressure sensing catheter. Each of pressure sensing device 104 and pressure sensing device 106 can be disposed in the vessel of the patient during a pre-PCI procedure. Further, in some examples, processor 110 can execute instructions 120 to receive distal pressure 122 and aortic pressure 124 in the absence of a hyperemic agent.

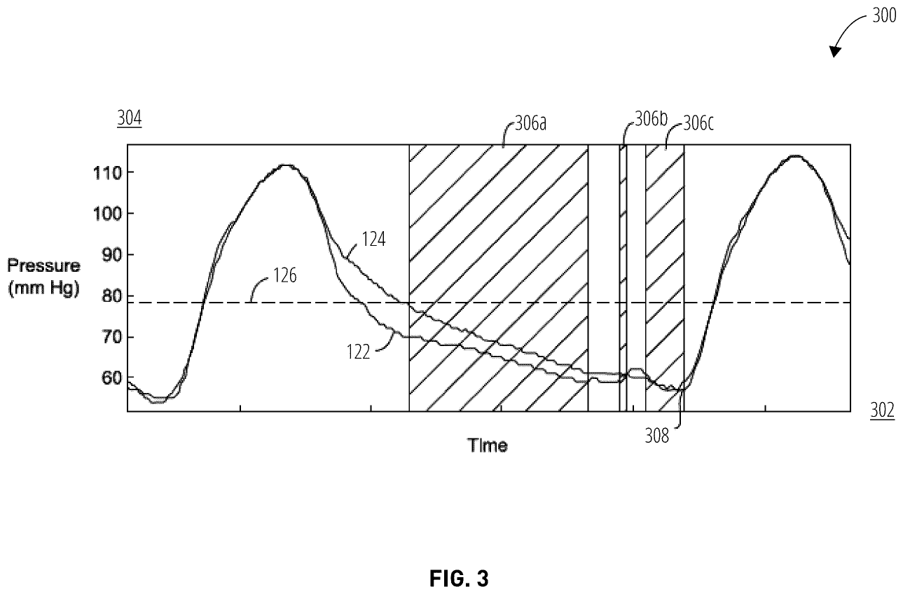

Continuing to block 206 “calculate a slope of a plot of the second pressure over the first time period” a slope of a plot of the second pressure can be derived. FIG. 3 illustrates plot 300, which graphically depicts pressure measurements over several cardiac cycles (e.g., one full cycle is depicted plus a portion of another cardiac cycle). Plot 300 depicts distal pressure 122 and aortic pressure 124 plotted against time on the x axis 302 and shown in the measured pressure in millimeters of Mercury (mm Hg) on the y axis 304. Further, plot 300 shows aortic pressure mean 126 over time. During operation (e.g., during a pre-PCI procedure, or the like) processor 110 can execute instructions 120 to derive a slope of aortic pressure 124.

Continuing to block 208 “calculate a mean of the second pressure over the first time period” a mean of the second pressure is derived over the first period. Processor 110 can execute instructions 120 to derive aortic pressure mean 126 from aortic pressure 124. Plot 300 depicts aortic pressure mean 126.

Continuing to block 210 “identify one or more regions of within the first time period where the second pressure is less than or equal to the mean and where the slope of the plot of the second pressure is less than or equal to zero” a number of regions of the plot (e.g., plot 300, or the like) are identified wherein the regions comprise include where the second pressure is less than or equal to the mean of the second pressure and where the slope of the plot is negative. Processor 110 can execute instructions 120 to identify regions of a plot of aortic pressure 124 wherein the aortic pressure 124 is less than the aortic pressure mean 126 and where the slope of the plot is negative (e.g., less than or equal to zero). For example, plot 300 depicts regions 306a, 306b, and 306c, which correspond to where aortic pressure 124 is less than aortic pressure mean 126 and the slope of aortic pressure 124 is negative.

With some embodiments, processor 110 can execute instructions 120 to identify FFR regions 128 where aortic pressure 124 is less than aortic pressure mean 126 when aortic pressure mean 126 is scaled by a scaling factor and where the slope of aortic pressure mean 126 is negative. In some examples, the scaling factor is 0.5 to 1.5, 0.75 to 1.25, or 0.95 to 1.05.

With some embodiments, processor 110 can execute instructions 120 to identify FFR regions 128 where aortic pressure 124 is less than or equal to the aortic pressure mean 126 (or aortic pressure mean 126 scaled as described above) and greater than or equal to a lower bound of the aortic pressure mean 126. With some examples, the lower bound of the aortic pressure mean 126 can be derived as the aortic pressure mean 126 minus an offset (e.g., between 10 mmHg and 100 mmHg). In some examples, the lower bound of the aortic pressure mean 126 can be derived as the aortic pressure mean 126 times a scaling factor where the scaling factor is less than 1 (e.g., 0.8, 0.89, between 0.5 and 0.95, or the like). In some examples, the lower bound of the aortic pressure mean 126 can be derived as the aortic pressure mean 126 times a scaling factor where the scaling factor is less than 1 (e.g., 0.8, 0.89, between 0.5 and 0.95, or the like). In some examples, the lower bound of the aortic pressure mean 126 can be derived as the minimum 308 of the aortic pressure mean 126 plus a positive offset (e.g., between 10 mmHg and 100 mmHg).

Continuing to block 212 “calculate a plurality of fractional flow reserve (FFR) values as the ratio of the first pressure over the second pressure during the one or more regions” several FFR values can be derived as the ration of the first pressure over the second pressure during the identified regions. Processor 110 can execute instructions 120 to derive FFR values 130 from distal pressure 122 and aortic pressure 124 during FFR regions 128. Specifically, processor 110 can execute instructions 120 to derive FFR values 130 as distal pressure 122 over aortic pressure 124 during FFR regions 128 (e.g., regions 306a, 306b, and 306c).

Continuing to block 214 “identify for each of the plurality of FFR values, a maximum of the FFR value and n prior FFR values where n is greater than one” a maximum of the current FFR value and n prior FFR values is identified. For example, for each value of FFR values 130 the maximum of the value and n prior values can be identified where n is greater than one (1). In some embodiments, n can be between 3 and 5. In a specific embodiment, n can be 3. That is, processor 110 can execute instructions 120 to identify for each value of FFR values 130 the maximum of the value and the prior 3 values and store the maximum as FFR maximum values 132.

Continuing to block 216 “generate a trend line comprising the identified maximum FFR values” a trend line comprising the maximum identified values can be generated. For example, processor 110 can execute instructions 120 to plot FFR maximum values 132 as trend line 134.

With some embodiments, pressure measurement system 100 can derive trend line 134 for each individual cardiac cycle or for multiple cardiac cycles.

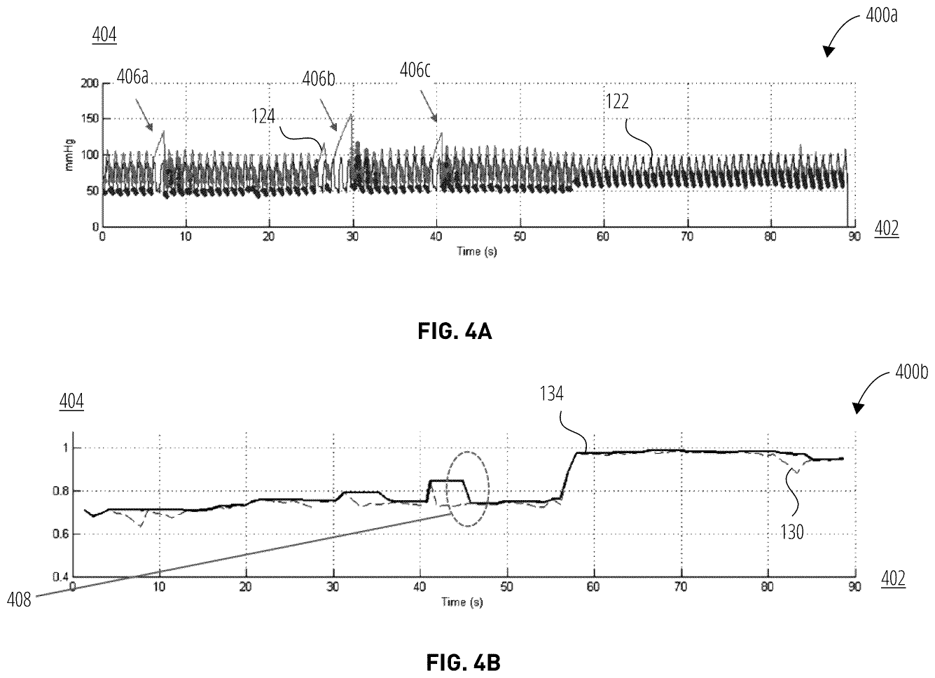

FIG. 4A illustrates plot 400a, which graphically depicts a flushing artifacts on aortic pressure during an FFR pullback. Plot 400a depicts aortic pressure 124 and aortic pressure mean 126 plotted over time on the x axis 402, where the y axis 404 shows the pressure at any given time. Further, this plot shows the effects of flushing on aortic pressure 124. For example, several flushing events 406a, 406b, and 406c are highlighted. As can be seen, each flushing event (e.g., flushing event 406a, flushing event 406b, and flushing event 406c) cause a spike in aortic pressure 124 but not distal pressure 122, which results in artifacts in FFR values 130. These artifacts make it challenging for a physician to analyze an FFR curve to identify areas where to treat a stenosis or lesion.

FIG. 4B illustrates plot 400b, which graphically depicts a trend line versus FFR values over several cardiac cycles and highlights the flushing events depicted in plot 400a. As can be seen, trend line 134 experienced a slight increase in measurement after flushing events 406a and 406b and a greater increase in measurement after flushing event 406c. However, trend line 134 returned to normal values (e.g., area 408) after several cardiac cycles (e.g., approximately 4 in this case) highlighting that the increase was due to an artifact and not a lesion or stenosis.

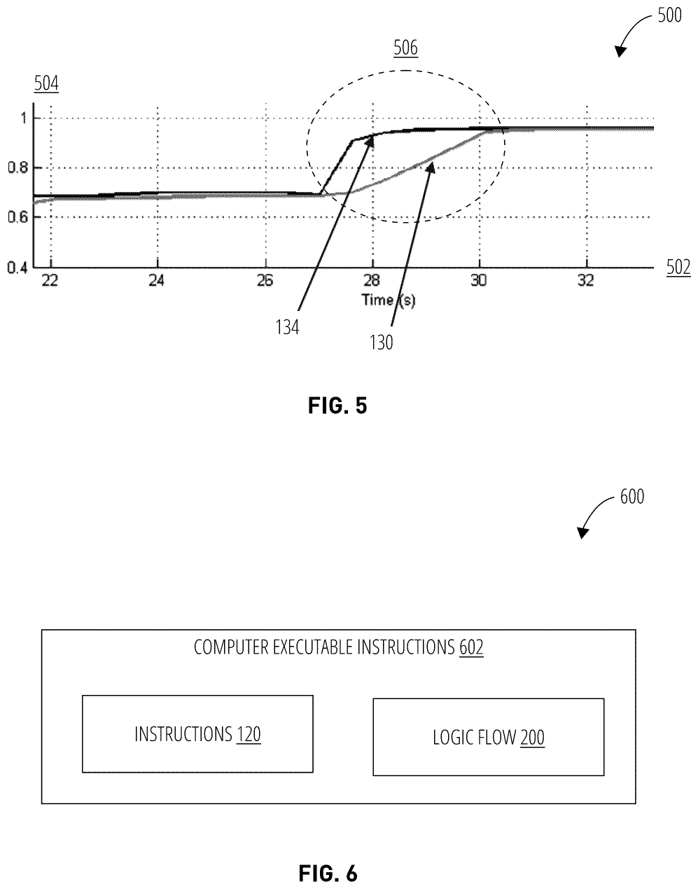

Further, as outlined above, trend line 134 provides a clearer indication of step ups in pressure resulting from lesions or stenosis. FIG. 5 illustrates plot 500, which graphically depicts a trend line versus FFR values over several cardiac cycles and a step up in pressure gradient, which can indicate a stenosis or lesion. For example, trend line 134 includes a sharper transition of “step up” in region 506 while FFR values 130 shows a more gradual change. The sharper transition in region 506 could increase the ability to distinguish a focal lesion from a diffuse distribution.

FIG. 6 illustrates computer-readable storage medium 600. Computer-readable storage medium 600 may comprise any non-transitory computer-readable storage medium or machine-readable storage medium, such as an optical, magnetic or semiconductor storage medium. In various embodiments, computer-readable storage medium 600 may comprise an article of manufacture. In some embodiments, computer-readable storage medium 600 may store computer executable instructions 602 with which circuitry (e.g., processor 110, pressure acquisition circuitry 118, and the like) can execute. For example, computer executable instructions 602 can include instructions to implement operations described with respect to instructions 120 and/or logic flow 200. Examples of computer-readable storage medium 600 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 602 may include any suitable type of code, such as source code, compiled code, interpreted code, executable code, static code, dynamic code, object-oriented code, visual code, and the like.

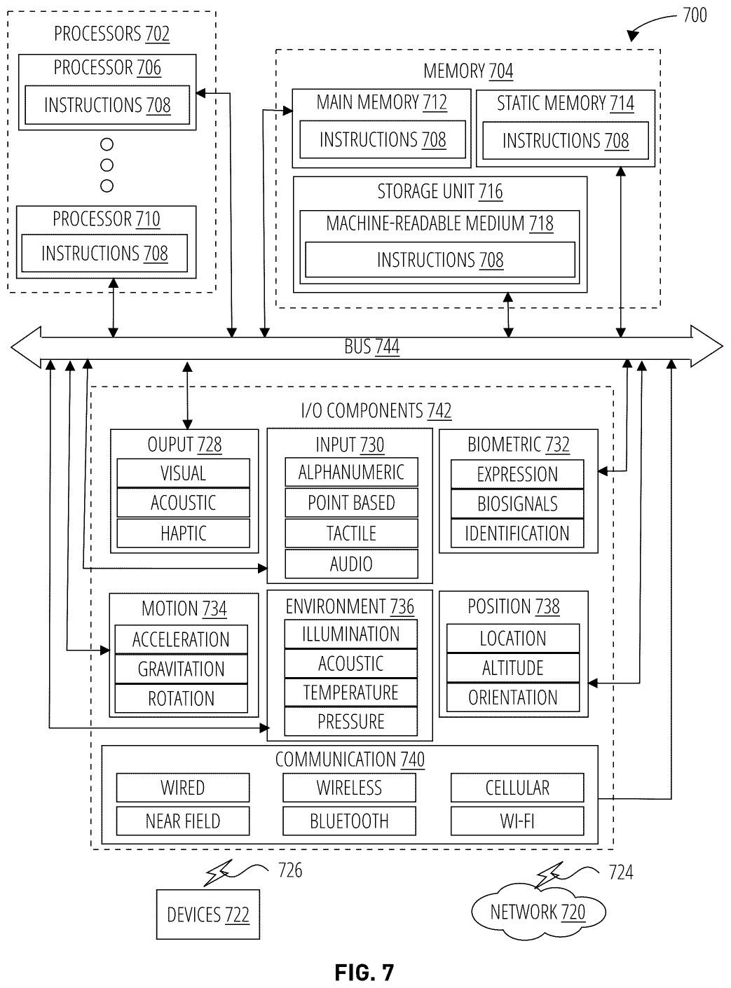

FIG. 7 illustrates a diagrammatic representation of a machine 700 in the form of a computer system within which a set of instructions may be executed for causing the machine to perform any one or more of the methodologies discussed herein. More specifically, FIG. 7 shows a diagrammatic representation of the machine 700 in the example form of a computer system, within which instructions 708 (e.g., software, a program, an application, an applet, an app, or other executable code) for causing the machine 700 to perform any one or more of the methodologies discussed herein may be executed. For example, the instructions 708 may cause the machine 700 to execute logic flow 200 of FIG. 2, or the like. More generally, the instructions 708 may cause the machine 700 to generate trend line 134 and/or GUI 138 including trend line 134 as described above.

The instructions 708 transform the general, non-programmed machine 700 into a particular machine 700 programmed to carry out the described and illustrated functions in a specific manner. In alternative embodiments, the machine 700 operates as a standalone device or may be coupled (e.g., networked) to other machines. In a networked deployment, the machine 700 may operate in the capacity of a server machine or a client machine in a server-client network environment, or as a peer machine in a peer-to-peer (or distributed) network environment. The machine 700 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 708, sequentially or otherwise, that specify actions to be taken by the machine 700. Further, while only a single machine 700 is illustrated, the term “machine” shall also be taken to include a collection of machines 700 that individually or jointly execute the instructions 708 to perform any one or more of the methodologies discussed herein.

The machine 700 may include processors 702, memory 704, and I/O components 742, which may be configured to communicate with each other such as via a bus 744. In an example embodiment, the processors 702 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 706 and a processor 710 that may execute the instructions 708. The term “processor” is intended to include multi-core processors that may comprise two or more independent processors (sometimes referred to as “cores”) that may execute instructions contemporaneously. Although FIG. 7 shows multiple processors 702, the machine 700 may include a single processor with a single core, a single processor with multiple cores (e.g., a multi-core processor), multiple processors with a single core, multiple processors with multiples cores, or any combination thereof.

The memory 704 may include a main memory 712, a static memory 714, and a storage unit 716, both accessible to the processors 702 such as via the bus 744. The main memory 704, the static memory 714, and storage unit 716 store the instructions 708 embodying any one or more of the methodologies or functions described herein. The instructions 708 may also reside, completely or partially, within the main memory 712, within the static memory 714, within machine-readable medium 718 within the storage unit 716, within at least one of the processors 702 (e.g., within the processor's cache memory), or any suitable combination thereof, during execution thereof by the machine 700.

The I/O components 742 may include a wide variety of components to receive input, provide output, produce output, transmit information, exchange information, capture measurements, and so on. The specific I/O components 742 that are included in a particular machine will depend on the type of machine. For example, portable machines such as mobile phones will likely include a touch input device or other such input mechanisms, while a headless server machine will likely not include such a touch input device. It will be appreciated that the I/O components 742 may include many other components that are not shown in FIG. 7. The I/O components 742 are grouped according to functionality merely for simplifying the following discussion and the grouping is in no way limiting. In various example embodiments, the I/O components 742 may include output components 728 and input components 730. The output components 728 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 730 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 742 may include biometric components 732, motion components 734, environmental components 736, or position components 738, among a wide array of other components. For example, the biometric components 732 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 734 may include acceleration sensor components (e.g., accelerometer), gravitation sensor components, rotation sensor components (e.g., gyroscope), and so forth. The environmental components 736 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 738 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 742 may include communication components 740 operable to couple the machine 700 to a network 720 or devices 722 via a coupling 724 and a coupling 726, respectively. For example, the communication components 740 may include a network interface component or another suitable device to interface with the network 720. In further examples, the communication components 740 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 722 may be another machine or any of a wide variety of peripheral devices (e.g., a peripheral device coupled via a USB).

Moreover, the communication components 740 may detect identifiers or include components operable to detect identifiers. For example, the communication components 740 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 740, such as location via Internet Protocol (IP) geolocation, location via Wi-Fi® signal triangulation, location via detecting an NFC beacon signal that may indicate a particular location, and so forth.

The various memories (i.e., memory 704, main memory 712, static memory 714, and/or memory of the processors 702) and/or storage unit 716 may store one or more sets of instructions and data structures (e.g., software) embodying or utilized by any one or more of the methodologies or functions described herein. These instructions (e.g., the instructions 708), when executed by processors 702, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 720 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 720 or a portion of the network 720 may include a wireless or cellular network, and the coupling 724 may be a Code Division Multiple Access (CDMA) connection, a Global System for Mobile communications (GSM) connection, or another type of cellular or wireless coupling. In this example, the coupling 724 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 708 may be transmitted or received over the network 720 using a transmission medium via a network interface device (e.g., a network interface component included in the communication components 740) and utilizing any one of several well-known transfer protocols (e.g., hypertext transfer protocol (HTTP)). Similarly, the instructions 708 may be transmitted or received using a transmission medium via the coupling 726 (e.g., a peer-to-peer coupling) to the devices 722. The terms “transmission medium” and “signal medium” mean the same thing and may be used interchangeably in this disclosure. The terms “transmission medium” and “signal medium” shall be taken to include any intangible medium that can store, encoding, or carrying the instructions 708 for execution by the machine 700, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

By using genuine models of anatomy more accurate surgical plans may be developed than through statistical modeling.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A system for determining fractional flow reserve (FFR) in the absence of administering a hyperemic agent, the system comprising:
a memory device comprising instructions; and
a processor coupled to the memory device, the processor configured to execute the instructions, which when executed cause the system to:
receive an indication of a first pressure from a pressure sensing guidewire coupled to the system for a first time period, wherein the first time period is during a medical intervention and wherein the first pressure is measured within a vessel of a patient;
receive an indication of a second pressure from a pressure sensing medical device different from the pressure sensing guidewire for the first time period, wherein the second pressure is measured within the vessel of the patient;
calculate a slope of a plot of the second pressure over the first time period;
calculate a mean of the second pressure over the first time period;
identify one or more regions within the first time period where the second pressure is less than or equal to the mean of the second pressure and where the slope of the plot of the second pressure is less than or equal to zero;
calculate a plurality of FFR values as the ratio of the first pressure over the second pressure during the one or more regions;
identify for each of the plurality of FFR values, a maximum of the FFR value and n prior FFR values where n is greater than one; and
generate a trend line comprising the identified maximum FFR values.

2. The system of claim 1, wherein n is greater than or equal to 3.

3. The system of claim 1, wherein n is greater than or equal to 3 and less than or equal to 5.

4. The system of claim 1, the memory device further comprising instructions that when executed by the processor cause the system to identify the one or more regions within the first time period where the second pressure is less than or equal to a factor of the mean.

5. The system of claim 4, wherein the factor is between 0.75 and 0.85.

6. The system of claim 5, wherein the factor is 0.8.

7. The system of claim 1, wherein the pressure sensing guidewire comprises an optical pressure sensor.

8. The system of claim 1, wherein the pressure sensing medical device comprises a catheter with a pressure sensor.

9. The system of claim 1, wherein the pressure sending guidewire and/or the pressure sensing medical device are wirelessly coupled to the system.

10. The system of claim 1, comprising the pressure sending medical device and the pressure sensing guidewire.

11. The system of claim 1, wherein the pressure sensing guidewire is configured to be disposed in the vessel distal of an intravascular lesion.

12. The system of claim 11, wherein the pressure sensing medical device is configured to be disposed in the vessel proximal of the intravascular lesion.

13. The system of claim 1, the memory device further comprising instructions that when executed by the processor cause the system to generate a graphical user interface comprising a graphical representation of the trend line.

14. The system of claim 13, the memory device further comprising instructions that when executed by the processor cause the system to render the graphical user interface for display on a display.

15. The system of claim 14, comprising the display.

16. At least one machine readable storage device, comprising a plurality of instructions that in response to being executed by a processor of a hemodialysis machine cause the processor to:
receive an indication of a first pressure from a pressure sensing guidewire coupled to the system for a first time period, wherein the first time period is during a medical intervention and wherein the first pressure is measured within a vessel of a patient;
receive an indication of a second pressure from a pressure sensing medical device different from the pressure sensing guidewire for the first time period, wherein the second pressure is measured within the vessel of the patient;
calculate a slope of a plot of the second pressure over the first time period;
calculate a mean of the second pressure over the first time period;
identify one or more regions within the first time period where the second pressure is less than or equal to the mean of the second pressure and where the slope of the plot of the second pressure is less than or equal to zero;
calculate a plurality of FFR values as the ratio of the first pressure over the second pressure during the one or more regions;
identify for each of the plurality of FFR values, a maximum of the FFR value and n prior FFR values where n is greater than one; and
generate a trend line comprising the identified maximum FFR values.

17. The at least one machine readable storage device of claim 16, wherein the factor is between 0.75 and 0.85.

18. The at least one machine readable storage device of claim 16, wherein n is greater than or equal to 3.

19. A method, comprising:
receiving an indication of a first pressure from a pressure sensing guidewire coupled to the system for a first time period, wherein the first time period is during a medical intervention and wherein the first pressure is measured within a vessel of a patient;
receiving an indication of a second pressure from a pressure sensing medical device different from the pressure sensing guidewire for the first time period, wherein the second pressure is measured within the vessel of the patient;
calculating a slope of a plot of the second pressure over the first time period;
calculating a mean of the second pressure over the first time period;
identifying one or more regions within the first time period where the second pressure is less than or equal to the mean of the second pressure and where the slope of the plot of the second pressure is less than or equal to zero;
calculating a plurality of FFR values as the ratio of the first pressure over the second pressure during the one or more regions;
identifying for each of the plurality of FFR values, a maximum of the FFR value and n prior FFR values where n is greater than one; and
generating a trend line comprising the identified maximum FFR values.

20. The method of claim 19, wherein n is greater than or equal to 3.
